(19) United States
(12) Patent Application Publication
(10) Pub. No.: US 2017/0105760 A1
(43) Pub. Date: Apr. 20, 2017

(54) CHEST TUBE INSERTION GUIDE AND METHOD OF USING THE SAME

(71) Applicant: PRISCILLA FLAGLER, WEST PALM BEACH, FL (US)

(72) Inventor: PRISCILLA FLAGLER, WEST PALM BEACH, FL (US)

(21) Appl. No.: 14/884,091

(22) Filed: Oct. 15, 2015

(51) Int. Cl. A61B 17/34; A61M 27/00

(52) U.S. Cl. CPC A61B 17/3415; A61M 27/00; A61M 2210/101; A61B 2017/3488

(57) ABSTRACT

The chest tube insertion guide includes a hollow cylindrical body having opposed proximal and distal ends, with a conical tip formed on the proximal end thereof. The conical tip is divided into a plurality of segments by axially-extending lines of separation, such that adjacent ones of the plurality of segments are frangibly joined to one another at the lines of separation. In use, an incision is created in a patient's chest above a pleural space to be drained of fluid. The conical tip of the chest tube insertion guide is inserted into the incision and an outer lining of the lung of the patient is punctured with the conical tip of the chest tube. The chest tube is inserted into the hollow cylindrical body of the chest tube insertion guide and is used to separate the plurality of segments of the cylindrical tip of the chest tube insertion guide.

### BACKGROUND OF THE INVENTION

1. Field of the Invention

The present invention relates to chest tube insertion and placement, and particularly to a chest tube insertion guide to be used in combination with a chest tube for guiding insertion thereof into a pleural space of a patient's lung to be drained of fluid.

2. Description of the Related Art

A chest tube insertion involves the surgical placement of a hollow, non-flexible drainage tube into the chest. Chest tubes are used to drain blood, fluid or air and to allow the lung(s) to fully expand. The tube is placed between the ribs and into the space between the inner lining and the outer lining of the lung (i.e., the pleural space). The area where the tube will be inserted is typically numbed using local anesthesia, although sedation may also be used. As shown in FIG. 2A, an incision I is first made in the anesthetized area, which is located above the pleural space of interest; i.e., above the region where blood, air or other fluid has built up between the inner lining and outer lining of the patient's lung L. In the particular example of FIG. 2A, a 2-3 cm incision I is made at the fifth intercostal space, anterior to the mid-axial line, although it should be understood that this particular placement is shown for exemplary purposes only.

As shown in FIG. 2B, the chest tube T is inserted through the incision I, between the ribs R, and into the chest, specifically with the proximal end P of the chest tube T positioned within the pleural space PS defined between the outer lining OL and the inner lining IL of the lung L. The distal end of chest tube T (not shown) is typically connected to a bottle or canister that contains sterile water. Suction is then attached to the system for drainage. As shown, a clamp C is typically used to hold the proximal end P of the chest tube T in place. A stitch (suture) and adhesive tape is typically used to keep the tube T in place during drainage of pleural fluid F.

The chest tube T usually stays in place until x-rays or the like show that all the blood, fluid or air has drained from the chest and the lung L has fully re-expanded. When the chest tube T is no longer needed, it is removed from the patient. At the point after the surgeon has created incision I but before insertion of the chest tube T, it is common for the surgeon to manually open the outer lining OL of the lung L; i.e., the surgeon may pierce and hold open the fragile outer lining OL with his finger. This not only requires the surgeon to effectively lose the usage of one of his hands during the tube insertion, but also removes a degree of accuracy in the procedure due to potential human error. Thus, a chest tube insertion guide and method of using the same solving the aforementioned problems is desired.

### SUMMARY OF THE INVENTION

The chest tube insertion guide includes a hollow cylindrical body having opposed proximal and distal ends, with a conical tip formed on the proximal end thereof. The conical tip is divided into a plurality of segments by axially-extending lines of separation, such that adjacent ones of the plurality of segments are frangibly joined to one another at the axially-extending lines of separation.

In use, an incision is created in a patient's chest above a pleural space to be drained of fluid. The conical tip of the chest tube insertion guide is inserted into the incision and an outer lining of the lung of the patient, above the pleural space to be drained of the fluid. The proximal end of the chest tube is inserted into the distal end of the hollow cylindrical body of the chest tube insertion guide and is used to separate the plurality of segments of the cylindrical tip of the chest tube insertion guide. This positions the proximal end of the chest tube in the pleural space to be drained of the fluid. Following insertion of the chest tube, the chest tube insertion guide is then removed from the incision and slid over the chest tube, away from the patient. The chest tube may then be secured using sutures and tape, as is conventionally known.

It should be understood that the chest tube insertion guide may be provided on its own, or may be provided to the user in the form of a kit including the chest tube insertion guide and the chest tube. It should be further understood that such a kit may also include any suitable associated items, such as sutures, adhesive tape and the like

These and other features of the present invention will become readily apparent upon further review of the following specification and drawings.

### BRIEF DESCRIPTION OF THE DRAWINGS

FIG. 1A is a perspective view of a chest tube insertion guide according to the present invention, shown in an initial closed configuration.

FIG. 1B is a perspective view of the chest tube insertion guide of FIG. 1A, shown in a final open configuration.

FIG. 2A illustrates an initial incision step in a conventional, prior art chest tube insertion procedure.

FIG. 2B illustrates insertion and clamping of the chest tube in the conventional, prior art chest tube insertion procedure.

FIG. 3A illustrates the chest tube insertion guide of FIG. 1A inserted into a patient's chest and into a pleural space of the patient's lung, prior to insertion of a chest tube therethrough.

FIG. 3B illustrates the chest tube being inserted into the patient's chest using the chest tube insertion guide of FIG. 1A.

FIG. 3C illustrates removal of the chest tube insertion guide of FIG. 1A following insertion of the chest tube into the patient's chest.

Similar reference characters denote corresponding features consistently throughout the attached drawings.

### DETAILED DESCRIPTION OF THE PREFERRED EMBODIMENTS

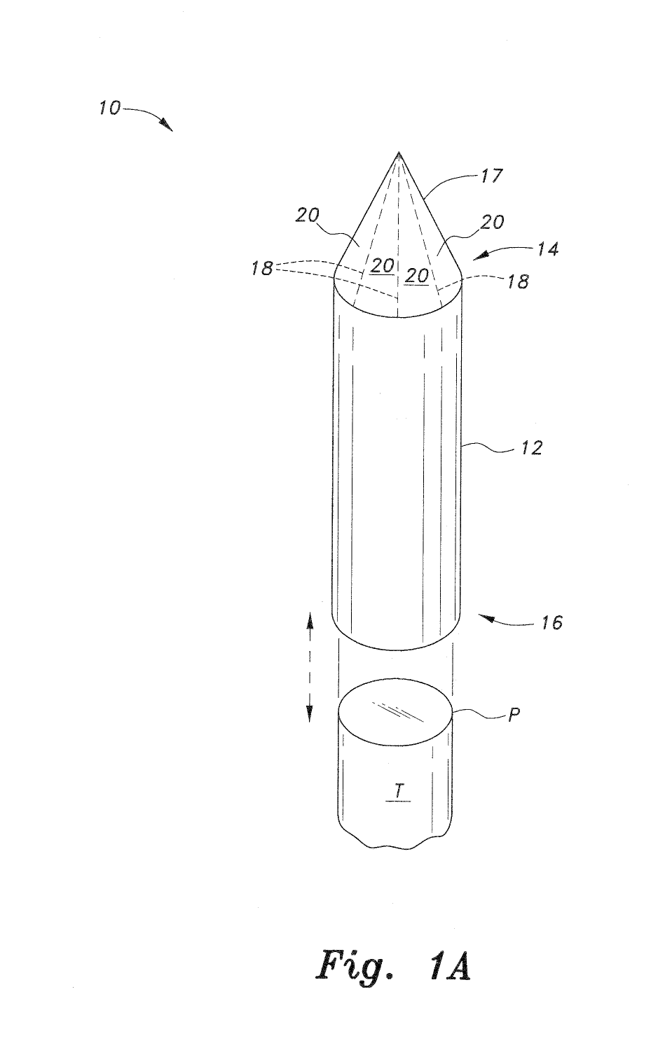

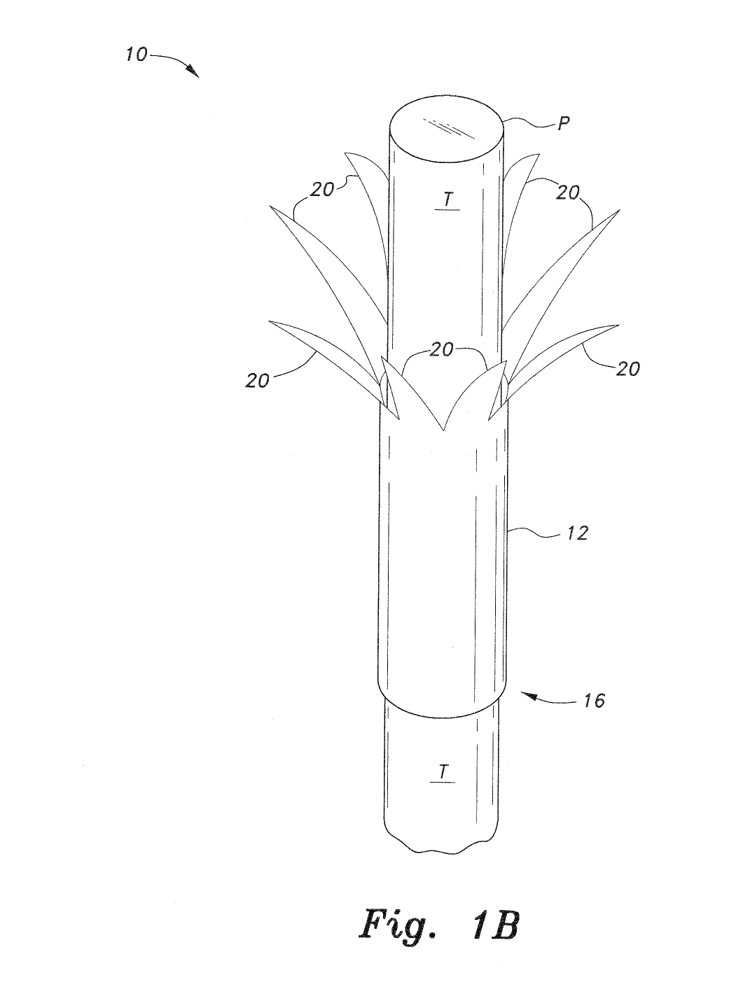

Referring now to FIG. 1A, the chest tube insertion guide 10 includes a hollow cylindrical body 12 having opposed proximal and distal ends, 14, 16, respectively, with a conical tip 17 being formed on the proximal end 14. As shown, the conical tip 17 is divided into a plurality of segments 20 by axially-extending lines of separation 18, such that adjacent ones of the plurality of segments 20 are frangibly joined to one another at the axially-extending lines of separation 18. It should be understood that frangible lines of separation 18 may be in the form of any suitable type of frangible connection, such as score lines, cuts, perforations or the like. It should also be understood that the chest tube insertion guide 10 may be made from any suitable material, such as sterile, surgical grade plastic or the like.

It should be further understood that the relative dimensions and overall contouring of chest tube insertion guide 10 are shown for exemplary purposes only, and that chest tube insertion guide 10 may have any desired relative dimensions and overall contouring suitable for receiving a chest tube and, as will be described in greater detail below, for puncturing a thin fascia layer between the ribs, above a pleural space PS around the lungs L to be drained of fluid F. As shown in FIG. 1B, chest tube insertion guide 10 is sized and shaped to receive chest tube T such that the proximal end P thereof may be used to separate the plurality of segments 20. It should be understood that the chest tube insertion guide 10 may be manufactured in a variety of sizes for matching and receiving a variety of different sizes and styles of chest tubes.

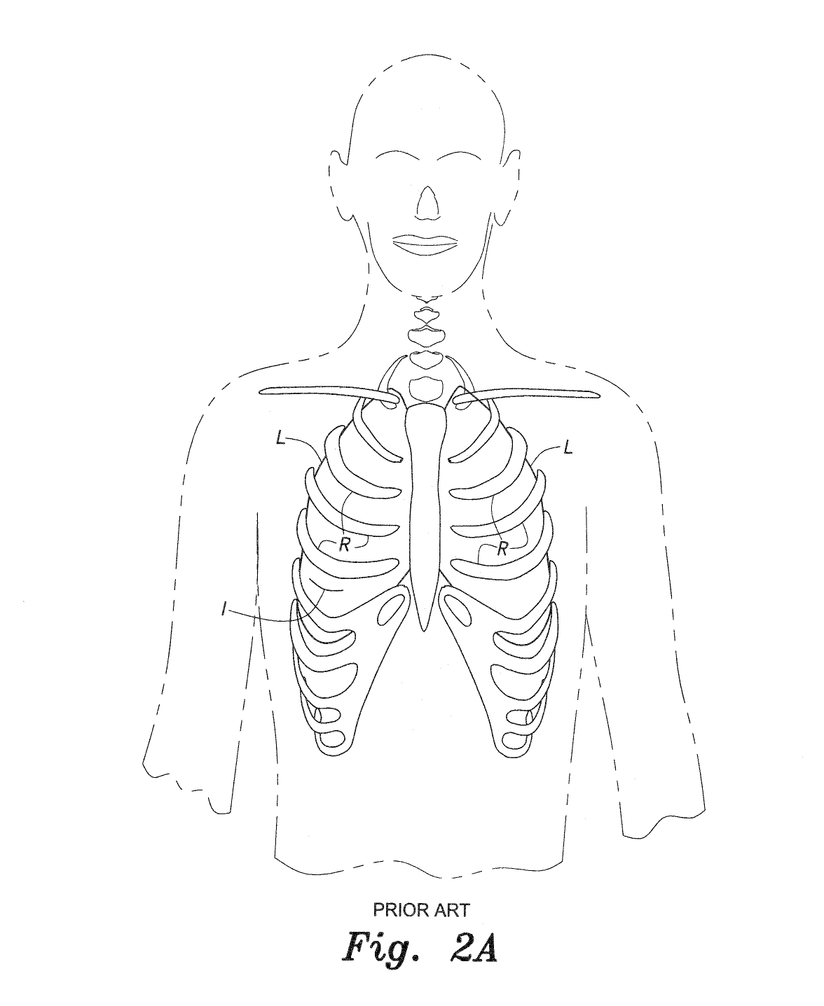

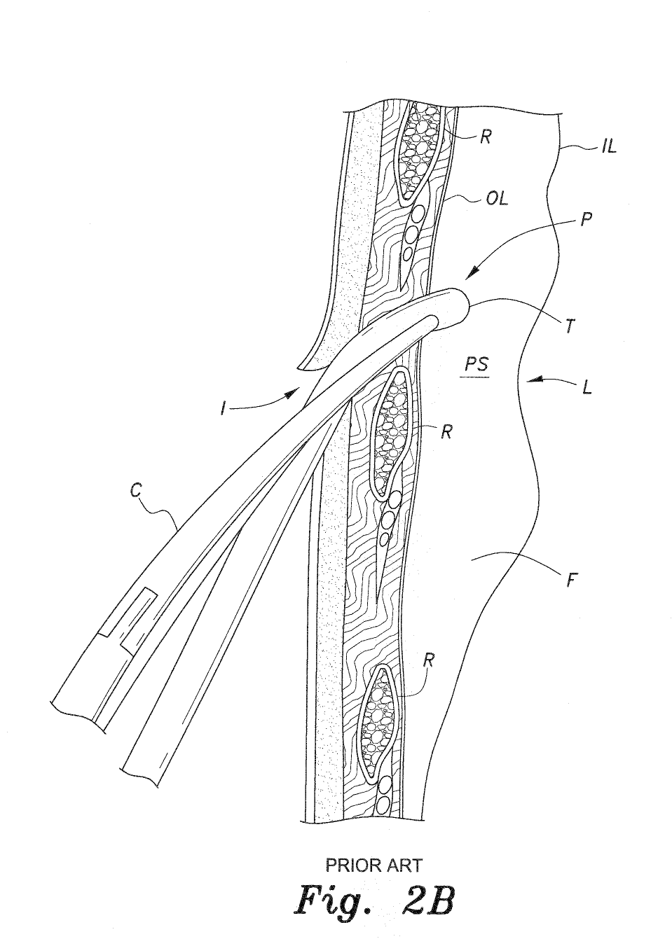

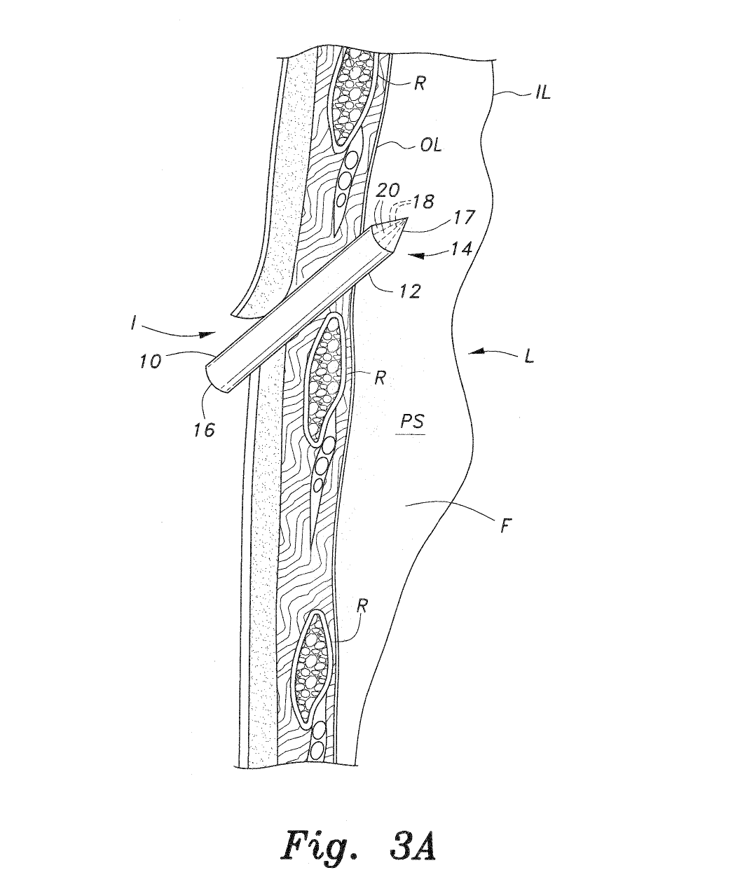

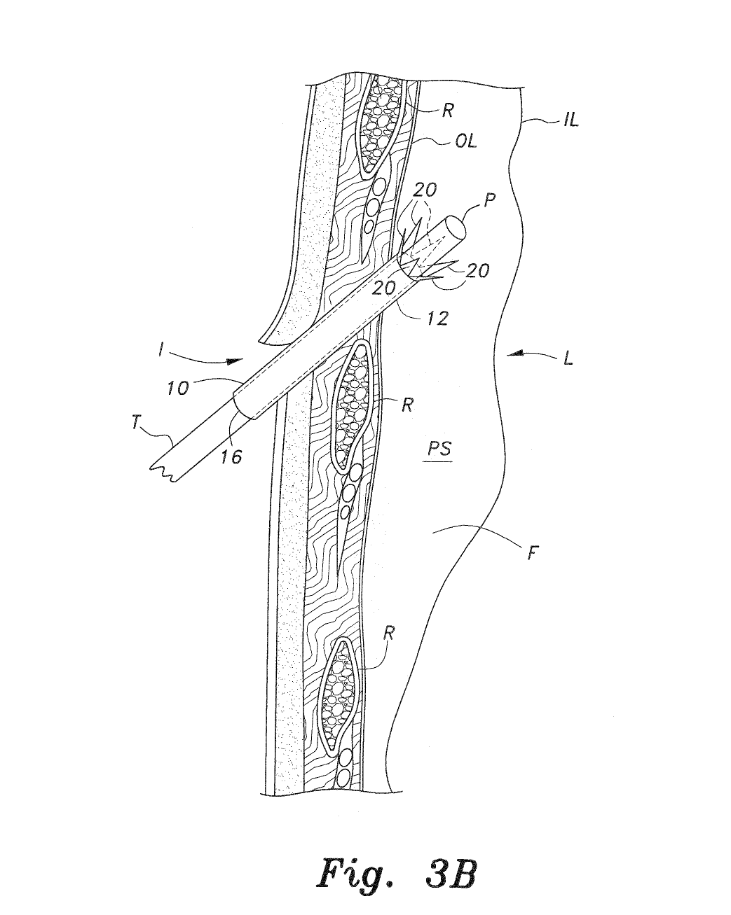

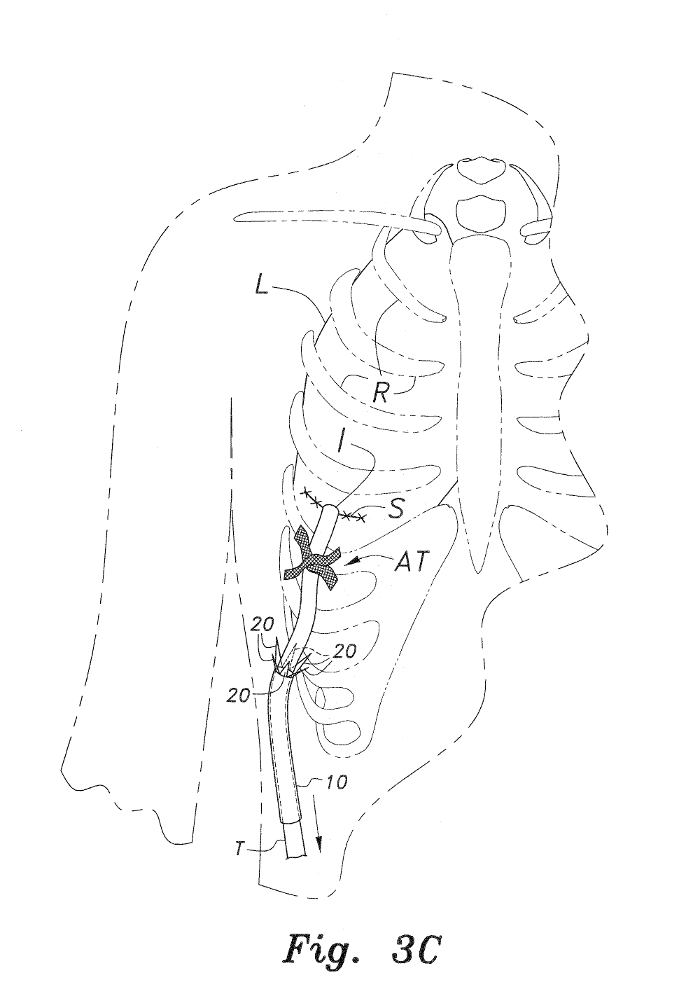

In use, an incision I is created in a patient's chest, above a pleural space PS to be drained of fluid F, as described above with respect to FIG. 2A. As shown in FIG. 3A, the conical tip 17 of the chest tube insertion guide 10 is then inserted into the incision I and the fascia layer or outer lining OL of the lung L of the patient, above the pleural space PS to be drained of the fluid F, is punctured with the conical tip 17. As shown in FIG. 3B, the proximal end P of the chest tube T is inserted into the distal end 16 of the hollow cylindrical body 12 of the chest tube insertion guide 10 and is used to separate the plurality of segments 20 of the cylindrical tip 17. This positions the proximal end P of the chest tube T in the pleural space PS to be drained of the fluid F. As shown in FIG. 3C, following insertion of the chest tube T, the chest tube insertion guide 10 is removed from the incision I and slid over the chest tube T, away from the patient. The chest tube T may then be secured using sutures S and adhesive tape AT, as is conventionally known.

It should be understood that the chest tube insertion guide 10 may be provided on its own, or may be provided to the user in the form of a kit including the chest tube insertion guide 10 and the chest tube T. It should be further understood that such a kit may also include any suitable associated items, such as sutures S, adhesive tape AT and the like.

It is to be understood that the present invention is not limited to the embodiments described above, but encompasses any and all embodiments within the scope of the following claims.

I claim:

1. A chest tube insertion guide, comprising:
a hollow cylindrical body having opposed proximal and distal ends, a conical tip being formed on the proximal end thereof, the conical tip being divided into a plurality of segments by axially-extending lines of separation, wherein adjacent ones of said plurality of segments are frangibly joined to one another at said axially-extending lines of separation,
whereby an incision may be created in a patient's chest above a pleural space to be drained of fluid such that the conical tip of the chest tube insertion guide may be inserted into the incision to puncture an outer lining of a lung of the patient above the pleural space to be drained of the fluid, a proximal end of a chest tube being inserted into the distal end of the hollow cylindrical body of the chest tube insertion guide such that the plurality of segments of the cylindrical tip of the chest tube insertion guide may be separated with the proximal end of the chest tube for positioning of the proximal end of the chest tube in the pleural space to be drained of the fluid.

2. A method of inserting a chest tube, comprising the steps of:
providing a chest tube insertion guide, the chest tube insertion guide comprising a hollow cylindrical body having opposed proximal and distal ends, a conical tip being formed on the proximal end thereof, the conical tip being divided into a plurality of segments by axially-extending lines of separation, wherein adjacent ones of said plurality of segments are frangibly joined to one another at said axially-extending lines of separation;
creating an incision in a patient's chest above a pleural space to be drained of fluid;
inserting the conical tip of the chest tube insertion guide into the incision;
puncturing an outer lining of a lung of the patient above the pleural space to be drained of the fluid with the conical tip of the chest tube insertion guide;
inserting a proximal end of a chest tube into the distal end of the hollow cylindrical body of the chest tube insertion guide;
separating the plurality of segments of the cylindrical tip of the chest tube insertion guide with the proximal end of the chest tube; and
positioning the proximal end of the chest tube in the pleural space to be drained of the fluid.

3. The method of inserting a chest tube as recited in claim 2, further comprising the steps of:
removing the chest tube insertion guide from the incision; and
sliding the chest tube insertion guide over the chest tube, away from the patient.

4. A chest intubation kit, comprising:
a chest tube; and
a chest tube insertion guide comprising a hollow cylindrical body having opposed proximal and distal ends, a conical tip being formed on the proximal end thereof, the conical tip being divided into a plurality of segments by axially-extending lines of separation, wherein adjacent ones of said plurality of segments are frangibly joined to one another at said axially-extending lines of separation,
whereby an incision may be created in a patient's chest above a pleural space to be drained of fluid such that the conical tip of the chest tube insertion guide may be inserted into the incision to puncture an outer lining of a lung of the patient above the pleural space to be drained of the fluid, a proximal end of the chest tube being inserted into the distal end of the hollow cylindrical body of the chest tube insertion guide such that the plurality of segments of the cylindrical tip of the chest tube insertion guide may be separated with the proximal end of the chest tube for positioning of the proximal end of the chest tube in the pleural space to be drained of the fluid.
